(19) United States
(12) Patent Application Publication
(10) Pub. No.: US 2016/0243192 A1
(43) Pub. Date: Aug. 25, 2016

(54) ANTIBODY DEPENDENT EXOSOME THERAPY

(71) Applicant: CHILDREN'S HOSPITAL LOS ANGELES, Los Angeles, CA (US)

(72) Inventors: Robert C. SEEGER, Los Angeles, CA (US); Muller FABBRI, Los Angeles, CA (US); Ambrose JONG, Arcadia, CA (US); Alan S. WAYNE, Los Angeles, CA (US)

(73) Assignee: Children's Hospital Los Angeles, Los Angeles, CA (US)

(21) Appl. No.: 15/029,395

(22) PCT Filed: Oct. 17, 2014
(86) PCT No.: PCT/US14/61245

§ 371 Date: Apr. 14, 2016

Related U.S. Application Data
(60) Provisional application No. 61/892,352, filed on Oct. 17, 2013.
(60) Provisional application No. 62/038,734, filed on Aug. 18, 2014.

(51) Int. Cl. A61K 38/17; A61K 39/395; A61K 45/06; C07K 16/30

(52) U.S. Cl. CPC A61K 38/177; C07K 16/3053; A61K 39/39558; A61K 45/06; C07K 16/3061

(57) ABSTRACT

Described herein are methods, pharmaceutical compositions and kits for treating cancer in a subject in need thereof by administering an effective amount of a composition comprising exosomes that comprise Fc receptors. In some embodiments, the exosomes in the pharmaceutical compositions described herein comprise Fc receptors including Fcgamma1 (CD64), Fcgamma2 (CD32), Fcgamma3 (CD16) or combinations thereof.

### GOVERNMENT RIGHTS

The invention was made with government support under Grant No. CA081403-14 awarded by the National Cancer Institute and Grant No. 047599-09 awarded by the National Institutes of Health. The government has certain rights to the invention.

### FIELD OF INVENTION

Described herein are compositions and methods for treating cancer using compositions comprising exosomes for antibody dependent exosome therapy (ADET).

### BACKGROUND OF THE INVENTION

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Exosomes are extracellular biological nanovesicles that deliver active molecules (mRNAs, miRNAs, proteins etc.) to recipient cells and influence their functions. They can be involved in the pathogenesis of cancer and degenerative diseases. Exosomes appear to be vectorized signaling systems operating between the cytoplasm of a donor cell and either the extracellular compartment or potentially all the internal compartments of a target cell. There is a need in the art for effective targeting and therapeutic approaches to treat malignant diseases such as cancer and non-malignant diseases such as infections. Described herein are compositions comprising exosomes for antibody dependent exosome therapy and methods of using the same to treat malignant and non-malignant diseases.

### SUMMARY OF THE INVENTION

The following embodiments and aspects thereof are described and illustrated in conjunction with systems, compositions and methods which are meant to be exemplary and illustrative, not limiting in scope.

Described herein are pharmaceutical compositions and kits comprising exosomes isolated from cells and a pharmaceutically acceptable carrier.

In some embodiments, the exosomes in the pharmaceutical compositions described herein comprise Fc receptors including FcγR1(CD64), FcγR2(CD32), FcγR3(CD16) or combinations thereof. In an embodiment, the exosomes comprise Fc receptors including FcγR1(CD64), FcγR3(CD16) or combinations thereof. In an embodiment, the exosomes comprise FcγR1(CD64).

In some embodiments, the exosomes are natural as described herein (for example, exosomes secreted by activated natural killer (aNK) cells) and comprise Fc receptors. In various embodiments, the exosomes are derived from any cell that naturally produces exosomes with Fc receptors. In an exemplary embodiment, the exosomes are derived from aNK cells.

In some embodiments, the exosomes are engineered as described herein (for examples, exosomes released by cells that are modified to express Fc receptors) and comprise Fc receptors. In various embodiments, the exosomes are derived from cells that are engineered to produce exosomes with Fc receptors. In exemplary embodiments, exosomes comprising Fc receptors are derived from engineered T cells, engineered macrophages, engineered stem cells, engineered mesenchymal stromal cells or combinations thereof.

In various embodiments, the exosomes (natural or engineered) may further comprise therapeutic agents including but not limited to antibodies, proteins, peptides, nucleic acids, small molecules drugs or combinations thereof. In some embodiments, the antibodies target the exosomes to the target cells (such as cancer cells). In some embodiments, the exosomes comprise antibodies that target the exosomes to the target cells and further comprise miRNAs so as to alter (increase or decrease) expression of genes of interest. In various embodiments, the antibodies are conjugated to therapeutic agents.

In an embodiment, the exosomes (natural or engineered) comprise Fc receptors and one or more antibodies that target the exosome to the target cell (such as a cancer cell) via the Fc receptors. In some embodiments, the exosomes comprise Fc receptors, one or more antibodies and one or more additional therapeutic agents. Therapeutic agents may include chemotherapeutic agents, immunomodulatory agents, anti-bacterial agents, anti-viral agents, anti-parasitic agents or combinations thereof. In various embodiments, the antibodies are conjugated to therapeutic agents.

In an exemplary embodiment, the exosomes in the compositions described herein are derived from aNK cells. These cells may be autologous or from healthy donors. The exosomes express Fc receptors such as FcγR1(CD64), FcγR2(CD32), FcγR3(CD16) or combinations thereof. In an embodiment, the exosomes derived from aNK cells comprise FcγR1(CD64). These exosomes may further comprise therapeutic agents including but not limited to chemotherapeutic agents, immunomodulatory agents, anti-bacterial agents, anti-viral agents, anti-parasitic agents or combinations thereof.

Described herein are methods for treating, inhibiting and/or reducing the severity of disease-states that are treatable by using the pharmaceutical compositions comprising the exosomes comprising Fc receptors (such as FcγR1(CD64), FcγR2(CD32), FcγR3(CD16) or combinations thereof) described herein. The methods include providing a composition comprising the exosomes comprising Fc receptors described herein and administering an effective amount of the composition to the subject in need thereof so as to treat the disease-state. In some embodiments, the disease-state is a metastatic disease such as cancer. In some embodiments, the disease-state is a non-metastatic disease such as infections, cardiac disease, autoimmune disease or combinations thereof. In some embodiments, the disease-state includes diseases caused by genetic deficiencies wherein the exosomes described herein deliver the missing gene product. In some embodiments, the disease-state is includes degenerative diseases in which the exosomes described herein deliver therapeutic molecules so as to block or reverse the degenerative process. In some embodiments, the compositions further comprise therapeutic agents including but not limited to chemotherapeutic agents, immunomodulatory agents, anti-bacterial agents, anti-viral agents, anti-parasitic agents or combinations thereof. In various embodiments, the therapeutic agents are therapeutic antibodies that are loaded into the exosomes described herein. In some embodiments, the antibodies are conjugated to therapeutic agents.

Further described herein are methods for treating cancer, inhibiting cancer, preventing metastasis of cancer, promoting remission of cancer and/or reducing the likelihood of cancer relapse in a subject in need thereof. The methods include providing a composition comprising the exosomes comprising Fc receptors described herein and administering an effective amount of the composition to the subject in need thereof so as to treat cancer, inhibit cancer, prevent cancer metastasis, promote cancer remission and/or reduce the likelihood of cancer relapse in a subject in need thereof. The exosomes in the compositions for treating cancer may be derived from aNK cells or any cell type that naturally produces exosomes comprising Fc receptors or are engineered to produce exosomes comprising Fc receptors. These cells may be autologous or from healthy donors. The exosomes express Fc receptors such as FcγR1(CD64), FcγR2(CD32), FcγR3(CD16) or combinations thereof. In an embodiment, the exosomes derived from aNK cells comprise FcγR1(CD64). In some embodiments, the compositions further comprise therapeutic agents including but not limited to chemotherapeutic agents, immunomodulatory agents or combinations thereof. In exemplary embodiments, the cancer is neuroblastoma or leukemia.

In exemplary embodiments, described herein are methods for treating, inhibiting, preventing metastasis of and/or promoting remission of neuroblastoma in a subject in need thereof. The methods include providing a composition comprising the exosomes comprising Fc receptors described herein and administering an effective amount of the composition to the subject in need thereof so as to treat, inhibit, prevent metastasis of, and/or promote remission of neuroblastoma in the subject. The exosomes in the compositions for treating neuroblastoma may be derived from aNK cells or any cell type that naturally produces exosomes comprising Fc receptors or is engineered to produce exosomes comprising Fc receptors. These cells may be autologous or from healthy donors. The exosomes express Fc receptors such as FcγR1(CD64), FcγR2(CD32), FcγR3(CD16) or combinations thereof. In an embodiment, the exosomes derived from aNK cells comprise FcγR1(CD64). These exosomes may further comprise therapeutic agents including but not limited to chemotherapeutic agents, immunomodulatory agents, anti-bacterial agents, anti-viral agents, anti-parasitic agents or combinations thereof. In some embodiment, the therapeutic agents are therapeutic antibodies that may further be conjugated to additional therapeutic agents.

In exemplary embodiments, described herein are methods for treating, inhibiting, preventing metastasis of and/or promoting remission of leukemia (such as acute lymphoblastic leukemia (ALL), acute myeloid leukemia (AML), chronic lymphocytic leukemia (CLL), juvenile myelomonocytic leukemia (JMML) and chronic myeloid leukemia (CML)) in a subject in need thereof. The methods include providing a composition comprising the exosomes comprising Fc receptors described herein and administering an effective amount of the composition to the subject in need thereof so as to treat, inhibit, prevent metastasis of, and/or promote remission of leukemia in the subject. The exosomes in the compositions for treating leukemia may be derived from aNK cells or any cell type that naturally produces exosomes comprising Fc receptors or is engineered to produce exosomes comprising Fc receptors. These cells may be autologous or from healthy donors. The exosomes express Fc receptors such as FcγR1(CD64), FcγR2(CD32), FcγR3(CD16) or combinations thereof. In an embodiment, the exosomes derived from aNK cells comprise FcγR1(CD64). These exosomes may further comprise therapeutic agents including but not limited to chemotherapeutic agents, immunomodulatory agents, anti-bacterial agents, anti-viral agents, anti-parasitic agents or combinations thereof. In some embodiment, the therapeutic agents are therapeutic antibodies that may further be conjugated to additional therapeutic agents. In some embodiment, the therapeutic agents are therapeutic antibodies that may further be conjugated to additional therapeutic agents.

Also provided herein are methods determining the efficacy of antibody dependent exosome therapy comprising determining the targeting efficacy of the exosomes to the target cells and/or tissues. In some embodiments, determining the targeting efficacy of the exosomes to the target cells and/or tissues includes detecting in the target cells and/or tissues the nucleic acids and/or proteins that are specific to the exosomes. The presence of exosome-specific nucleic acids and/or proteins in the target cells and/or tissues is indicative of effective antibody dependent exosome therapy. In some embodiments, the exosomes are derived from activated natural killer cells. In some embodiments, the exosomes are engineered to include specific proteins and/or nucleic acids that are unique to the exosomes. In some embodiments, nucleic acids include miRNAs, mRNAs or a combination thereof. In exemplary embodiments, the miRNAs may be any one or more of the miRNAs set forth in Table 4. In further exemplary embodiments, the mRNAs may be any one or more of the mRNAs set forth in Table 5.

### BRIEF DESCRIPTION OF FIGURES

Exemplary embodiments are illustrated in the referenced figures. It is intended that the embodiments and figures disclosed herein are to be considered illustrative rather than restrictive.

FIG. 1 depicts, in accordance with various embodiments of the present invention, release of exosomes by NK cells, antibody dependent cellular cytotoxicity (ADCC), and antibody dependent exosome cytotoxicity (ADEC) mediated by exosomes released from NK cells. A) Activated NK cells release exosomes that can be cytotoxic for NB cells. B) NK cell mediated ADCC. C) aNK exosomes express Fc receptors for IgG (CD64, demonstrated by mass spectrometry and Western blotting and CD16, demonstrated by ExoFlow and NanoSight analysis). The binding of mAbs such as ch14.18 enhances binding and cytotoxicity of aNK exosomes for NB cells mediating “ADEC”.

FIG. 2 depicts, in accordance with various embodiments of the present invention, ex vivo expansion of aNK cells and large-scale aNK exosome isolation. (A) Flow chart for exosome isolation using a proprietary polymer precipitation method. Aliquots of isolated exosomes were diluted with PBS (1:100) and subjected to NanoSight particle analysis. (B) A representative chart of the mean particle size 155 nm and (C) an image of light reflective particles are shown. (D) Summary of mean particle size for different isolation methods. Overall, the three methods are comparable, but our precipitation method is readily scaled to larger volumes for GMP preparations, is efficient, and is inexpensive.

FIG. 3 depicts, in accordance with various embodiments of the present invention, that activated NK cells derived exosomes express CD64. Western Blot analysis: ten μg of proteins, from either (1) isolated exosomes or (2) NK cell lysates, was used for the Western blot. The protein band was detected by anti-CD64 antibody (1:500, R&D Systems, Inc; cat. # MAB12571.

FIG. 4 depicts, in accordance with various embodiments of the present invention, that aNK exosomes increase cytotoxicity of aNK cells. CHLA-255Fluc and CHLA-136-Fluc are neuroblastoma cell lines.

FIG. 5 depicts, in accordance with various embodiments of the present invention that aNK exosomes are cytotoxic for NB cell lines and aNK-derived exosomes are cytotoxic for neuroblastoma cells in NOD/SCID mice. 10⁶ CHLA-255-Fluc cells (neuroblastoma cells) were injected in 25% low growth factor Matrigel in both shoulders of 4 mice. On days 8 and 10, NK exosomes (175 μg) were injected into the left tumor area, whereas a control solution (10% glycerol in PBS) was injected in the contralateral tumor area. Mice were imaged pre-treatment (day 7; grey bars) and post-treatment (days 11; black bars). Bar graph shows average, SD, and t test P values for bioluminescence of 4 tumors in each group. At day 21, 6/8 control tumors were ≧3 mm diameter whereas none of the treated tumors were palpable.

FIG. 6 depicts, in accordance with various embodiments of the present invention, cytotoxicity of aNK exosomes against neuroblastoma (CHLA255-Luc; grey bars) cells and ALL (SupB15-Luc; black bars) cells. The percentage is the survival percentage.

FIG. 7 depicts, in accordance with various embodiments of the present invention, suppression of MYCN and SMAD2/3 protein expression by miR-16 and miR-186 in human neuroblastoma (CHLA-136 and CHLA-255) cell lines. 100 nM miR-16 and miR-186 were transfected into the cells using DOTAP transfection.

FIG. 8 depicts, in accordance with various embodiments of the present invention, uptake and cytotoxicity of aNK exosomes for NB cells is increased by mAb ch14.18. A) aNK exosome (white arrows, FM6-4 stain) uptake by CHLA-255-Fluc NB cells (grey, DAPI stain) is increased by ch14.18. Control Cryptococcus exosomes are not taken up. B) Cytotoxicity of aNK exosomes (mg/ml) for CHLA-255-Fluc cells is increased by anti-GD2 mAB ch14.18 (24 hr assay; acridine orange/propidium iodide stain; Luna FL instrument). C) Cytotoxicity determined with 7AAD/Annexin-V flow cytometry assay (24 hr assay; exosomes, 0.5 mg/ml; ch14.18, 1 μg/ml).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

For references on pediatrics, see Schwartz et al., The 5-Minute Pediatric Consult 4^{th} ed., Lippincott Williams & Wilkins, (Jun. 16, 2005); Robertson et al., The Harriet Lane Handbook: A Manual for Pediatric House Officers 17^{th} ed., Mosby (Jun. 24, 2005); and Hay et al., Current Diagnosis and Treatment in Pediatrics (Current Pediatrics Diagnosis & Treatment) 18^{th} ed., McGraw-Hill Medical (Sep. 25, 2006).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer. In some embodiments, the disease condition is an autoimmune disease.

“Cancer” and “cancerous” refer to or describe the physiological condition in mammals that is typically characterized by unregulated cell growth. Examples of cancer include, but are not limited to lymphomas (Hodgkin's lymphomas and/or non-Hodgkin's lymphomas), sarcomas, brain cancer, breast cancer, colon cancer, lung cancer, hepatocellular cancer, gastric cancer, pancreatic cancer, cervical cancer, ovarian cancer, leukemia, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Chemotherapeutic drugs” or “chemotherapeutic agents” as used herein refer to drugs used to treat cancer including but not limited to Albumin-bound paclitaxel (nab-paclitaxel), Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Lapatinib, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, Ixabepilone, Estramustine, or a combination thereof.

“Chimeric antigen receptor” or “CAR” or “CARs” as used herein refers to engineered receptors, which graft an antigen specificity onto cells (for example T cells such as naïve T cells, central memory T cells, effector memory T cells or combination thereof). CARs are also known as artificial T-cell receptors, chimeric T-cell receptors or chimeric immunoreceptor.

“Subject” or “individual” or “animal” or “patient” or “mammal,” is meant any subject, particularly a mammalian subject, for whom diagnosis, prognosis, or therapy is desired. In some embodiments, the subject has cancer. In some embodiments, the subject had cancer at some point in the subject's lifetime. In various embodiments, the subject's cancer is in remission, is re-current or is non-recurrent.

“Mammal” as used herein refers to any member of the class Mammalia, including, without limitation, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject. The term does not denote a particular age or sex. Thus, adult and newborn subjects, as well as fetuses, whether male or female, are intended to be included within the scope of this term.

“Treatment” and “treating,” as used herein refer to therapeutic treatment or prophylactic measures or preventative measures or combinations thereof, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Examples of cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or a combination thereof.

“Tumor,” as used herein refers to all neoplastic cell growth and proliferation, whether malignant or benign, and all pre-cancerous and cancerous cells and tissues.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases.

Exosomes are extracellular biological nanovesicles secreted by many cell types including but not limited to Natural Killer (NK) cells, dendritic cells, T cells, macrophages, stem cells and mesenchymal stromal cells and cancer cells. Activated natural killer (aNK) cells are potent killers of infected and/or cancer cells. They may exert this function by releasing exosomes that deliver cytotoxic molecules such as perforin and granzyme B to target cells, which cause cell death. The inventors have reported that highly cytotoxic NK cells from patients can be grown in vitro and that these aNK cells release large quantities of exosomes while they are growing in vitro. In characterizing the exosomes released from aNK, the inventors discovered that these exosomes naturally have Fc receptors (such as CD64/FcγR1 and CD16/FcγR3 receptors) that bind immunoglobulin (IgG) antibodies (for example, anti-GD2 antibody ch14.18). CD64 has not previously been described to be on either NK cells or their exosomes. It was further discovered that an IgG antibody (ch14.18) that is used clinically to treat neuroblastoma increases exosome binding and entry into neuroblastoma cells. Therefore, while not wishing to be limited to any particular hypothesis, it is believed that the binding of this or other anti-cancer antibodies to CD64 and/or CD16 on exosomes will markedly enhance targeting of cancer cells to mediate cytotoxicity or other effects. In essence, via the Fc receptors on the exosomes, a therapeutic IgG antibody can target and mediate uptake of exosomes by cancer cells; this is termed “antibody dependent exosome therapy” (ADET). ADET will be generally applicable for cytotoxic therapy against, for example, malignant cells, non-malignant cells (e.g., virus infected) and even against microorganisms when specific antibodies are available.

Additionally, cells that do not express the Fc receptors (such as CD64/FcγR1 or CD16/FcγR3) could be genetically engineered to express the receptors and so engender their exosomes with the ability to bind antibodies, thus allowing targeting of exosomes from a variety of cells including but not limited to mesenchymal stromal cells, dendritic cells, regulatory T cells, macrophages and/or stem cells for the purpose of regulating functions of the targeted cells rather than destroying them through cytotoxicity.

Accordingly, described herein are methods for producing exosomes, compositions comprising exosomes and methods for therapeutically using exosomes to treat disease states including but not limited to cancer, non-malignant diseases or combination thereof.

### Compositions

Provided herein are pharmaceutical compositions comprising exosomes comprising Fc receptors and a pharmaceutically acceptable carrier. The compositions include exosomes derived from cells that naturally express Fc receptors or cells that are genetically engineered to express Fc receptor. The Fc receptors may be any one or more of FcγR1(CD64), FcγR2(CD32), FcγR3(CD16) or combinations thereof. Examples of cells that naturally express the Fc receptors include but are not limited to activated natural killer (aNK) cells. Examples of cells that may be genetically engineered to express the Fc receptors include but are not limited to T cells, macrophages, stem cells, mesenchymal stem cells or combinations thereof. The compositions comprising exosomes comprising Fc receptors may further comprise therapeutic agents including but not limited to antibodies, proteins, peptides, nucleic acids, small molecules drugs or combinations thereof. In an embodiment, the exosomes comprise Fc receptors and one or more antibodies that target the exosome to the target cell (such as a cancer cell) via the Fc receptors. In some embodiments, the exosomes comprise Fc receptors, one or more antibodies and one or more additional therapeutic agents. Therapeutic agents may include chemotherapeutic agents, immunomodulatory agents, anti-bacterial agents, anti-viral agents, anti-parasitic agents or combinations thereof.

As described herein, in some embodiments, the exosomes may be “natural” exosomes that comprise Fc receptors. Natural exosomes are obtained by isolating cells that naturally express Fc receptors and expanding the cells in culture. Natural exosomes are secreted by these cells and comprise Fc receptors. The cells may be autologous or from a healthy donor. The Fc receptors may be any one or more of FcγR1(CD64), FcγR2(CD32), FcγR3(CD16) or combinations thereof. In various embodiments, the natural exosomes may further comprise therapeutic agents including but not limited to antibodies, proteins, peptides, nucleic acids, small molecules drugs or combinations thereof. In some embodiments, the natural exosomes further comprise antibodies specific to an antigen expressed on target cells so as to target the exosomes to the target cells via the Fc receptors on the exosomes.

In an exemplary embodiment, natural exosomes are obtained by isolating natural killer cells, activating the isolated NK cells and expanding the activated cells in culture. aNK cells may express Fc receptors. The expanded aNK cells secrete exosomes which also comprise Fc receptors. In some embodiments, since the aNK cells are potent killers of cancer cells, the ex vivo expanded aNK cell are administered to the subject in an effective amount to be used with the methods described herein, such as to treat cancer (for example, neuroblastoma or ALL). Further, since the NK cells are potent terminators of cancer cells, the exosomes from ex vivo expanded aNK cells may also be effective in destroying cancer cells. In some embodiments, the natural exosomes are administered in an effective amount to be used with the methods described herein such as to treat cancer (for example, neuroblastoma or ALL). The aNK cells may be autologous or from a healthy donor. The Fc receptors on aNK cells and exosomes derived from aNK cells may be any one or more of FcγR1(CD64), FcγR2(CD32), FcγR3(CD16) or combinations thereof. The exosomes derived from aNK cells may further comprise therapeutic agents including but not limited to antibodies, proteins, peptides, nucleic acids, small molecules drugs or combinations thereof. In some embodiments, the natural exosomes derived from aNK cells may further comprise antibodies specific to an antigen expressed on target cells so as to target the exosomes to the target cells via the Fc receptors on the exosomes.

In some embodiments, the exosomes may be “engineered” exosomes. Engineered exosomes may be obtained by modifying (engineering; redirecting) exosome producing cells that do not naturally express for example, Fc receptors, to express Fc receptors and expanding the cells in culture. The engineered exosomes are secreted by these engineered cells and comprise Fc receptors. The cells may be autologous or obtained from healthy donors. The Fc receptors may be any one or more of FcγR1(CD64), FcγR2(CD32), FcγR3(CD16) or combinations thereof. In some embodiments, exosomes may be obtained by modifying exosomes producing cells to produce molecules that target antigens on surface of target cells (such as cancer cells). In various embodiments, the engineered exosomes may further comprise therapeutic agents including but not limited to antibodies, proteins, peptides, nucleic acids, small molecules drugs or combinations thereof. In some embodiments, the engineered exosomes further comprise antibodies specific to an antigen expressed on target cells so as to target the exosomes to the target cells via the Fc receptors on the exosomes.

In an exemplary embodiment, the engineered exosomes are secreted by engineered aNK cells. For example, the aNK cells may be modified (engineered; redirected) to express biological molecules (for example, nucleic acids or proteins) that are not ordinarily expressed by the aNK cells. In some embodiments, the ex vivo expanded engineered aNK cell are administered to the subject in an effective amount to be used with the methods described herein, such as to treat cancer (for example, neuroblastoma or ALL). The exosomes secreted by the engineered aNK cells are engineered exosomes. In some embodiments, the engineered exosomes are administered in an effective amount to be used with the methods described herein such as to treat cancer (for example, neuroblastoma or ALL). The engineered exosomes may further comprise therapeutic agents including but not limited to antibodies, proteins, peptides, nucleic acids, small molecules drugs or combinations thereof. The engineered aNK cells may be autologous or from a healthy donor. The Fc receptors on engineered aNK cells and exosomes derived from engineered aNK cells may be any one or more of FcγR1(CD64), FcγR2(CD32), FcγR3(CD16) or combinations thereof. The exosomes derived from engineered aNK cells may further comprise therapeutic agents including but not limited to antibodies, proteins, peptides, nucleic acids, small molecules drugs or combinations thereof. In some embodiments, the engineered exosomes derived from engineered aNK cells may further comprise antibodies specific to an antigen expressed on target cells so as to target the exosomes to the target cells via the Fc receptors on the exosomes.

Genetically modified cells may be produced by stably transfecting cells with DNA encoding the biological molecules of interest into the cells obtained from the subject (autologous) or from a healthy donor. The genetically modified cells may be subsequently expanded. Various methods produce stable transfectants which express the biological molecules of interest. In one embodiment, a method of stably transfecting and re-directing cells is by electroporation using naked DNA. By using naked DNA, the time required to produce engineered/redirected cells may be significantly reduced. Additional methods to genetically engineer cells using naked DNA encoding the biological molecules of interest include but are not limited to chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). The transfected cells demonstrating presence of the molecules of interest may be expanded ex vivo. Viral transduction methods may also be used to generate genetically modified cells. In various exemplary embodiments, the cells may be any one or more of NK cells, T cells, macrophages, stem cells, mesenchymal stromal cells or combinations thereof. In some embodiments, the cells are autologous. In some embodiments, the cells are obtained from healthy donors.

In additional embodiments, engineered exosomes may be obtained by first isolating exosomes secreted by the natural ex vivo expanded cells and subsequently loading the isolated exosomes with agents of interest. Agents of interest may be targeting agents, therapeutic agents or a combination thereof. Examples of therapeutic agents including but not limited to antibodies, proteins, peptides, nucleic acids, small molecules drugs or combinations thereof. Targeting agents target the ex vivo expanded cells or the exosomes to target cells (such as cancer cells, for example, neuroblastoma cells or leukemia (such as ALL)). In some embodiments, the targeting molecule is any one or more of lipids, peptides, proteins, antibodies, small molecules, oligonucleotides, nucleic acids or a combination thereof. In some embodiments, the therapeutic agent is any one or more of lipids, peptides, proteins, antibodies, small molecules, oligonucleotides, nucleic acids or a combination thereof.

In various embodiments, provided herein are pharmaceutical compositions including a pharmaceutically acceptable excipient and a therapeutically effective amount of any one or more of (i) ex vivo expanded cells that naturally produce Fc receptors, (ii) ex vivo expanded cells that are engineered to produce Fc receptors (iii) ex vivo expanded aNK cells, (iv) natural exosomes obtained from ex vivo expanded cells that naturally produce Fc receptors, (v) engineered exosomes obtained from ex vivo expanded cells that are engineered to produce Fc receptors, (vi) exosomes obtained from ex vivo expanded aNK cells, (vii) genetically modified exosomes (either obtained from ex vivo expanded autologous genetically modified cells or exosomes that are modified after obtaining exosomes from ex vivo expanded genetically modified cells), or combinations thereof, so as to treat cancer, prevent metastasis of cancer, reduce the severity of cancer, promote remission of cancer and/or prevent or reduce the likelihood of cancer relapse. In some embodiments, the cancer is neuroblastoma. In some embodiments, the cancer is ALL or other leukemia subtypes.

In some embodiments, the pharmaceutical composition further comprises any one or more of a targeting molecule, one or more of a therapeutic agent or a combination thereof. In various embodiments, the targeting molecule and/or the therapeutic molecule is any one or more of lipids, peptides, proteins, antibodies, small molecules, oligonucleotides, nucleic acids or a combination thereof that recognizes a specific marker on the surface of cancer cells.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the antibodies are administered by injection, either intravenously or intraperitoneally. Methods for these administrations are known to one skilled in the art.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the compositions. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glycans. The saccharides or glycans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C₄ to C₈ hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added. In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Additionally, the compositions can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546 which are all hereby incorporated by reference in their entireties. Preferred polymers are polyoxyethylated polyols and polyethylene glycol (PEG). PEG is soluble in water at room temperature and in some embodiments, has an average molecular weight between 1000 and 40,000, between 2000 and 20,000, or between 3,000 and 12,000. In some embodiments, PEG has at least one hydroxy group, such as a terminal hydroxy group. The hydroxy group may be activated to react with a free amino group on the inhibitor. However, it will be understood that the type and amount of the reactive groups may be varied to achieve a covalently conjugated PEG/antibody of the present invention.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a molecular weight in the same range as PEG. The structure for POG is shown in Knauf et al., 1988, J. Bio. Chem. 263:15064-15070 and a discussion of POG/IL C 2 conjugates is found in U.S. Pat. No. 4,766,106, both of which are hereby incorporated by reference in their entireties.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., Drug Delivery Systems (R. L. Juliano, ed., Oxford, N. Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

Controlled and Delayed Release Dosage Forms. In some embodiments of the methods described herein, the compositions comprising the exosomes described herein can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound's onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the compositions comprising the exosomes described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

In some embodiments, the compositions comprising the exosomes described herein for use in the methods described herein are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the compositions comprising the exosomes described herein can be administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

In various embodiments, the composition is administrated to the subject 1-3 times per day or 1-7 times per week. In various embodiments, the composition is administrated to the subject for 1-5 days, 1-5 weeks, 1-5 months, or 1-5 years.

An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent (exosomes described herein) which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The dosage and mode of administration will depend on the individual. Generally, the compositions are administered so that antibodies are given at a dose between 1 μg/kg and 20 mg/kg, between 20 μg/kg and 10 mg/kg, between 1 mg/kg and 7 mg/kg. In some embodiments, it is given as a bolus dose, to increase circulating levels by 10-20 fold and for 4-6 hours after the bolus dose. Continuous infusion may also be used after the bolus dose. If so, the antibodies may be infused at a dose between 5 μg/kg/minute and 20 μg/kg/minute, or between 7 μg/kg/minute and 15 μg/kg/minute.

### Methods

Also provided herein are methods for treating cancer, inhibiting cancer, preventing metastasis of cancer, reducing the severity of cancer, promoting remission of cancer and/or preventing or reducing the likelihood of cancer relapse in a subject in need thereof. The methods include providing a composition comprising exosomes comprising Fc receptors isolated from cells and administering an effective amount of the composition to the subject so as to treat cancer, inhibit cancer, prevent cancer metastasis, reduce the severity of cancer, promote remission of cancer and/or prevent or reduce the likelihood of cancer relapse in the subject. In various embodiments, the exosomes may be natural or engineered as described herein. In some embodiments, the exosomes comprise Fc receptors including any one or more of FcγR1(CD64), FcγR2(CD32), FcγR3(CD16) or combinations thereof. In an embodiment, the exosomes comprise Fc receptors including FcγR1(CD64), FcγR3(CD16) or combinations thereof. In one embodiment, the exosomes comprise FcγR1(CD64). In various embodiments, the exosomes (natural or engineered) may further comprise therapeutic agents including but not limited to antibodies, lipids, peptides, proteins, nucleic acids, small molecules or combinations thereof. In some embodiments, the compositions described herein are administered with additional therapeutic agents. In some embodiments, the exosomes are loaded with antibodies so as to target the exosomes to the cancer cells. In some embodiments, the antibodies are administered with the exosomes, either simultaneously or sequentially. In additional embodiments, the exosomes are administered with additional therapeutic (such as chemotherapeutic) agents. Therapeutic agents administered with the exosomes may be administered before, during or after the administration of the composition comprising the exosomes.

In various embodiments, the cancer may be any one or more of lymphomas, brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, leukemia, neuroblastoma and prostate cancer. In some embodiments, the composition further comprises one or more targeting molecules. In some embodiments, the composition further comprises one or more therapeutic agents. In additional embodiments, the composition further comprises one or more targeting molecules and one or more therapeutic agents. In an embodiment, composition further comprises an effective amount of ex vivo expanded autologous NK cells.

Also provided is a method for treating neuroblastoma, inhibiting neuroblastoma, preventing neuroblastoma metastasis, reducing the severity of neuroblastoma, promoting remission of neuroblastoma and/or preventing or reducing the likelihood of neuroblastoma relapse in a subject in need thereof. The methods include providing a composition comprising exosomes comprising Fc receptors isolated from cells and administering an effective amount of the composition to the subject so as to treat neuroblastoma, inhibit neuroblastoma, prevent neuroblastoma metastasis, reduce the severity of neuroblastoma, promote remission of neuroblastoma and/or prevent or reduce the likelihood of neuroblastoma relapse in the subject. In various embodiments, the exosomes may be natural or engineered as described herein. In some embodiments, the exosomes comprise Fc receptors including any one or more of FcγR1(CD64), FcγR2(CD32), FcγR3(CD16) or combinations thereof. In an embodiment, the exosomes comprise Fc receptors including FcγR1(CD64), FcγR3(CD16) or combinations thereof. In one embodiment, the exosomes comprise FcγR1(CD64). In various embodiments, the exosomes (natural or engineered) may further comprise therapeutic agents including but not limited to antibodies, lipids, peptides, proteins, nucleic acids, small molecules or combinations thereof. In some embodiments, the compositions described herein are administered with additional therapeutic agents. In some embodiments, the exosomes are loaded with antibodies so as to target the exosomes to the cancer cells. In some embodiments, the antibodies are administered with the exosomes, either simultaneously or sequentially. In additional embodiments, the exosomes are administered with additional therapeutic (such as chemotherapeutic) agents. Therapeutic agents administered with the exosomes may be administered before, during or after the administration of the composition comprising the exosomes. In an embodiment, the composition comprises exosomes derived from aNK cells wherein the exosomes comprise Fc receptors (for example, FcγR1(CD64), FcγR2(CD32), FcγR3(CD16) or combinations thereof) and an anti-GD2 antibody. In one embodiment, the cells are autologous. In another embodiment, the cells are from a healthy donor.

Also provided is a method for treating leukemia, inhibiting leukemia, preventing leukemia metastasis, reducing the severity of leukemia, promoting remission of leukemia and/or preventing or reducing the likelihood of leukemia relapse in a subject in need thereof. The methods include providing a composition comprising exosomes comprising Fc receptors isolated from cells and administering an effective amount of the composition to the subject so as to treat leukemia, inhibit leukemia, prevent leukemia metastasis, reduce the severity of leukemia, promote remission of leukemia and/or prevent or reduce the likelihood of leukemia relapse in the subject. In various embodiments, the exosomes may be natural or engineered as described herein. In some embodiments, the leukemia is ALL, AML, CLL, JMML or CML. In an embodiment, the leukemia is ALL. In some embodiments, the exosomes comprise Fc receptors including any one or more of FcγR1(CD64), FcγR2(CD32), FcγR3(CD16) or combinations thereof. In an embodiment, the exosomes comprise Fc receptors including FcγR1(CD64), FcγR3(CD16) or combinations thereof. In one embodiment, the exosomes comprise FcγR1(CD64). In various embodiments, the exosomes (natural or engineered) may further comprise therapeutic agents including but not limited to antibodies, lipids, peptides, proteins, nucleic acids, small molecules or combinations thereof. In some embodiments, the compositions described herein are administered with additional therapeutic agents. In some embodiments, the exosomes are loaded with antibodies so as to target the exosomes to the cancer cells. In some embodiments, the antibodies are administered with the exosomes, either simultaneously or sequentially. In additional embodiments, the exosomes are administered with additional therapeutic (such as chemotherapeutic) agents. Therapeutic agents administered with the exosomes may be administered before, during or after the administration of the composition comprising the exosomes. In an embodiment, the composition comprises exosomes derived from aNK cells wherein the exosomes comprise Fc receptors (for example, FcγR1(CD64), FcγR2(CD32), FcγR3(CD16) or combinations thereof) and an anti-CD19 antibody. In an embodiment, the composition comprises exosomes derived from aNK cells wherein the exosomes comprise Fc receptors (for example, FcγR1(CD64), FcγR2(CD32), FcγR3(CD16) or combinations thereof) and an anti-CD22 antibody. In an embodiment, the composition comprises exosomes derived from aNK cells wherein the exosomes comprise Fc receptors (for example, FcγR1(CD64), FcγR2(CD32), FcγR3(CD16) or combinations thereof) and an anti-CD19 and anti-CD22 antibody. In one embodiment, the cells are autologous. In another embodiment, the cells are from a healthy donor.

In various embodiments of the compositions and methods described herein, the exosomes comprise antibodies that may target antigens that are specific for cancer. Cancer specific antigens that may be targeted by the one or more targeting molecules include but are not limited to any one or more of disialoganglioside (GD2), 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD10, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, legumain, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, folic acid receptor, transferin receptors or combinations thereof. Other antigens specific for cancer will be apparent to those of skill in the art and may be used in connection with alternate embodiments of the invention.

As described herein, the exosomes comprising the Fc receptors may further comprise therapeutic antibodies. In some exemplary embodiments of the compositions and methods described herein, the antibodies may be any one or more of 3F8, 8H9, Abagovomab, Abciximab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab, Atorolimuma, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, cBR96-doxorubicin immunoconjugate, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, IMAB362, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab, Tildrakizumab, Tigatuzumab, TNX-650, Tocilizumab, Toralizumab, Tositumomab, Bexxar, Tovetumab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, Zolimomab aritox, or combinations thereof.

In some embodiments, the antibodies target the ex vivo expanded aNK cells (natural or engineered) or the exosomes (natural or engineered) to neuroblastoma cells. Targets on neuroblastoma cells include but are not limited to GD2, CD24, CD25 or a combination thereof.

In some embodiments, the antibodies target the ex vivo expanded aNK cells (natural or engineered) or the exosomes (natural or engineered) to ALL cells. Targets on ALL cells include but are not limited to CD2, CD10, CD15, CD19, CD21, CD22 or a combination thereof.

In some embodiments, exosomes comprising Fc receptors further comprise bispecific chimeric antigen receptors (CARs). These CARs are specific for at least two antigens on cancer cells and target the exosomes comprising Fc receptors to the cancer cells via the Fc receptors. In various embodiments the CARs target at least two or more antigens. Exemplary antigens targeted by the CARs include but are not limited to 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In some embodiments, the therapeutic agents are chemotherapeutic agents, that may be used with the methods described herein, such as to treat cancer including but are not limited to the chemotherapeutic agents described herein. In some embodiments, the therapeutic agents that may be used with the methods described herein, such as to treat cancer (for example, neuroblastoma or ALL) include but are not limited to anti-GD2 antibodies, 3F8 antibody, recombinant GM-CSF or a variant thereof, irinotecan/SN38, etoposide, fenretinide, PI-103, cyclophosamide, doxorubicin, therapeutic microRNAs (for example those that target oncogenes) or a combination thereof.

In some embodiments, the composition comprising engineered exosomes and one or more targeting molecules are separate from compositions comprising engineered exosomes and one or more therapeutic agents. If the two compositions are separate, the two compositions may be administered concurrently or separately. In other embodiments, the compositions comprising engineered exosomes also comprise one or more targeting molecules and one or more therapeutic agents.

In some embodiments, exosomes (natural or genetically engineered exosomes) comprise cancer-specific therapeutic micro RNAs including but not limited to the micro RNA listed in Table 1 (which refers to the -3p and the -5p mature sequence of the indicated microRNA).

TABLE 1
Cancer specific miRNAs.

| | | | | |
|---|---|---|---|---|
| hsa-miR-197 | hsa-miR-99b | hsa-miR-422a | hsa-miR-663b | hsa-miR-1272 |
| hsa-miR-23b | hsa-miR-30b | hsa-miR-375 | hsa-miR-325 | hsa-miR-20b |
| hsa-miR-1295 | hsa-miR-488* | hsa-miR-222 | hsa-miR-224 | hsa-miR-9 |
| hsa-miR-567 | hsa-miR-588 | hsa-miR-548d-5p | hsa-miR-875-3p | hsa-miR-1183 |
| hsa-miR-1268 | hsa-miR-1279 | hsa-miR-10a* | hsa-miR-181a* | hsa-miR-379* |
| hsa-miR-15b | hsa-miR-132 | hsa-miR-151-5p | hsa-miR-518e | hsa-miR-603 |
| hsa-miR-1246 | hsa-miR-185* | hsa-let-7a-1 | hsa-let-7a-2 | hsa-let-7a-3 |
| hsa-let-7e | hsa-let-7f-1 | hsa-let-7f-2 | hsa-let-7g | hsa-let-7i |
| hsa-mir-101-1 | hsa-mir-101-2 | hsa-mir-103-1 | hsa-mir-103-2 | hsa-mir-105-1 |
| hsa-mir-107 | hsa-mir-10a | hsa-mir-10b | hsa-mir-122 | hsa-mir-1226 |
| hsa-mir-1249 | hsa-mir-125a | hsa-mir-125b-1 | hsa-mir-125b-2 | hsa-mir-126 |
| hsa-mir-129-1 | hsa-mir-129-2 | hsa-mir-1291 | hsa-mir-1295 | hsa-mir-1296 |
| hsa-mir-132 | hsa-mir-133a-1 | hsa-mir-133a-2 | hsa-mir-133b | hsa-mir-134 |
| hsa-miR-107 | hsa-miR-767-3p | hsa-miR-422a | hsa-miR-1263 | hsa-miR-181a |
| hsa-mir-100 | hsa-mir-106b | hsa-mir-124-3 | hsa-mir-128-2 | hsa-mir-130b |
| hsa-let-7f | hsa-miR-662 | hsa-miR-325 | hsa-miR-1207-5p | hsa-miR-519a* |
| hsa-let-7c | hsa-mir-1-2 | hsa-mir-106a | hsa-mir-124-2 | hsa-mir-128-1 |
| hsa-let-7d | hsa-mir-135b | hsa-mir-136 | hsa-mir-137 | hsa-mir-138-1 |
| hsa-mir-139 | hsa-miR-139-5p | hsa-mir-140 | hsa-mir-141 | hsa-mir-142 |
| hsa-mir-144 | hsa-mir-145 | hsa-mir-146a | hsa-mir-146b | hsa-mir-147 |
| hsa-mir-148b | hsa-mir-149 | hsa-mir-150 | hsa-mir-151 | hsa-mir-152 |
| hsa-mir-153-2 | hsa-mir-154 | hsa-mir-155 | hsa-mir-15a | hsa-mir-15b |
| hsa-mir-16-2 | hsa-mir-17 | hsa-mir-181a-1 | hsa-mir-181a-2 | hsa-mir-181b-1 |
| hsa-mir-181c | hsa-mir-181d | hsa-mir-182 | hsa-mir-183 | hsa-mir-184 |
| hsa-mir-186 | hsa-mir-187 | hsa-mir-188 | hsa-mir-18a | hsa-mir-18b |
| hsa-mir-191 | hsa-mir-192 | hsa-mir-193a | hsa-mir-193a | hsa-mir-193b |
| hsa-mir-194-2 | hsa-mir-195 | hsa-mir-196a-1 | hsa-mir-196a-2 | hsa-mir-196b |
| hsa-mir-198 | hsa-mir-199a-1 | hsa-mir-199a-2 | hsa-mir-199b | hsa-mir-19a |
| hsa-mir-19b-2 | hsa-mir-200a | hsa-mir-200b | hsa-mir-200c | hsa-mir-202 |
| hsa-mir-204 | hsa-mir-205 | hsa-mir-206 | hsa-mir-208a | hsa-mir-20a |
| hsa-mir-21 | hsa-mir-210 | hsa-mir-211 | hsa-mir-212 | hsa-mir-214 |
| hsa-mir-216a | hsa-mir-217 | hsa-mir-218-1 | hsa-mir-218-2 | hsa-mir-219-1 |
| hsa-mir-22 | hsa-mir-220a | hsa-mir-221 | hsa-mir-222 | hsa-mir-223 |
| hsa-mir-23a | hsa-mir-23b | hsa-mir-24-1 | hsa-mir-24-2 | hsa-mir-25 |
| hsa-mir-26a-1 | hsa-mir-26a-2 | hsa-mir-26b | hsa-mir-27a | hsa-mir-27b |
| hsa-mir-296 | hsa-mir-299 | hsa-mir-29a | hsa-mir-29b-1 | hsa-mir-29b-2 |
| hsa-mir-301a | hsa-mir-302a | hsa-mir-302b | hsa-mir-302c | hsa-mir-302d |
| hsa-mir-30b | hsa-mir-30c-1 | hsa-mir-30c-2 | hsa-mir-30d | hsa-mir-30e |
| hsa-mir-32 | hsa-miR-32* | hsa-miR-320 | hsa-mir-320a | hsa-mir-323 |
| hsa-mir-325 | hsa-mir-326 | hsa-mir-328 | hsa-mir-329-1 | hsa-mir-329-2 |
| hsa-mir-331 | hsa-mir-335 | hsa-mir-337 | hsa-mir-338 | hsa-mir-339 |
| hsa-mir-33b | hsa-mir-340 | hsa-mir-342 | hsa-mir-345 | hsa-mir-346 |
| hsa-mir-34b | hsa-mir-34c | hsa-mir-361 | hsa-mir-362 | hsa-mir-363 |
| hsa-mir-365-2 | hsa-mir-367 | hsa-mir-369 | hsa-mir-370 | hsa-mir-371 |
| hsa-mir-373 | hsa-mir-374a | hsa-mir-374b | hsa-mir-375 | hsa-mir-376a-1 |
| hsa-mir-376c | hsa-mir-377 | hsa-mir-378 | hsa-mir-379 | hsa-mir-380 |
| hsa-mir-382 | hsa-mir-383 | hsa-mir-384 | hsa-mir-409 | hsa-mir-410 |
| hsa-mir-421 | hsa-mir-422a | hsa-mir-423 | hsa-mir-424 | hsa-mir-425 |
| hsa-mir-431 | hsa-mir-432 | hsa-mir-433 | hsa-mir-449a | hsa-mir-449b |
| hsa-mir-451 | hsa-mir-452 | hsa-mir-453 | hsa-mir-454 | hsa-mir-455 |
| hsa-mir-484 | hsa-mir-485 | hsa-mir-486 | hsa-mir-487a | hsa-mir-487b |
| hsa-mir-489 | hsa-mir-490 | hsa-mir-491 | hsa-mir-492 | hsa-mir-493 |
| hsa-mir-495 | hsa-mir-496 | hsa-mir-497 | hsa-mir-498 | hsa-mir-499 |
| hsa-mir-501 | hsa-mir-502 | hsa-mir-503 | hsa-mir-505 | hsa-mir-508 |
| hsa-mir-509-3 | hsa-mir-510 | hsa-mir-511-1 | hsa-mir-511-2 | hsa-mir-512-1 |
| hsa-mir-513a-1 | hsa-mir-513a-2 | hsa-mir-515-1 | hsa-mir-515-2 | hsa-mir-516a-1 |
| hsa-mir-516b-1 | hsa-mir-516b-2 | hsa-mir-517a | hsa-mir-517b | hsa-mir-517c |
| hsa-mir-518a-2 | hsa-mir-518b | hsa-mir-518c | hsa-mir-518d | hsa-mir-518e |
| hsa-mir-519a-1 | hsa-mir-519a-2 | hsa-mir-519b | hsa-mir-519c | hsa-mir-519d |
| hsa-mir-520a | hsa-mir-520b | hsa-mir-520c | hsa-mir-520d | hsa-mir-520e |
| hsa-mir-520g | hsa-mir-520h | hsa-mir-521-1 | hsa-mir-521-2 | hsa-mir-522 |
| hsa-mir-524 | hsa-mir-525 | hsa-mir-526a-1 | hsa-mir-526a-2 | hsa-mir-526b |
| hsa-mir-532 | hsa-mir-539 | hsa-mir-542 | hsa-mir-543 | hsa-mir-548o |
| hsa-mir-550-1 | hsa-mir-551a | hsa-mir-561 | hsa-mir-566 | hsa-mir-567 |
| hsa-mir-572 | hsa-mir-574 | hsa-mir-575 | hsa-mir-576 | hsa-mir-582 |
| hsa-mir-589 | hsa-mir-590 | hsa-mir-593 | hsa-mir-598 | hsa-mir-601 |
| hsa-mir-605 | hsa-mir-608 | hsa-mir-611 | hsa-mir-621 | hsa-mir-622 |
| hsa-mir-627 | hsa-mir-629 | hsa-mir-632 | hsa-mir-634 | hsa-mir-635 |
| hsa-mir-639 | hsa-mir-642 | hsa-mir-644 | hsa-mir-646 | hsa-mir-648 |
| hsa-mir-650 | hsa-mir-652 | hsa-mir-657 | hsa-mir-658 | hsa-mir-659 |
| hsa-mir-661 | hsa-mir-662 | hsa-mir-663 | hsa-mir-663b | hsa-mir-671 |
| hsa-mir-7-1 | hsa-mir-7-2 | hsa-mir-7-3 | hsa-mir-708 | hsa-mir-744 |
| hsa-mir-767 | hsa-miR-768-3p | hsa-mir-874 | hsa-mir-886 | hsa-mir-888 |
| hsa-miR-9 | hsa-mir-9-1 | hsa-mir-9-2 | hsa-mir-9-3 | hsa-mir-923 |
| hsa-mir-92a-2 | hsa-mir-92b | hsa-mir-93 | hsa-mir-95 | hsa-mir-96 |
| hsa-mir-99a | hsa-mir-99b | hsa-miR-99b* | mmu-mir-1-1 | mmu-mir-100 |
| mmu-mir-103-2 | mmu-mir-106a | mmu-mir-106b | mmu-mir-10a | mmu-mir-10b |
| mmu-mir-125b-1 | mmu-mir-126 | mmu-mir-130b | mmu-mir-133a-1 | mmu-mir-133b |
| mmu-mir-139 | mmu-mir-140 | mmu-mir-143 | mmu-mir-145 | mmu-mir-150 |
| mmu-mir-15a | mmu-mir-16-1 | mmu-mir-17 | mmu-mir-181a-1 | mmu-mir-181c |
| mmu-mir-184 | mmu-mir-18a | mmu-mir-192 | mmu-mir-193b | mmu-mir-196b |
| mmu-mir-19b-1 | mmu-mir-19b-2 | mmu-mir-203 | mmu-mir-207 | mmu-mir-20a |
| mmu-mir-210 | mmu-mir-22 | mmu-mir-222 | mmu-mir-223 | mmu-mir-23a |
| mmu-mir-25 | mmu-mir-27a | mmu-mir-27b | mmu-mir-290 | mmu-mir-292 |
| mmu-mir-298 | mmu-mir-29a | mmu-mir-29c | mmu-mir-301a | mmu-mir-30b |
| mmu-mir-31 | mmu-mir-322 | mmu-mir-33 | mmu-mir-346 | mmu-mir-351 |
| mmu-mir-365-1 | mmu-mir-375 | mmu-mir-378 | mmu-mir-450b | mmu-mir-466a |
| mmu-mir-468 | mmu-mir-486 | mmu-mir-500 | mmu-mir-503 | mmu-mir-542 |
| mmu-mir-669a-1 | mmu-mir-669c | mmu-mir-674 | mmu-mir-680-1 | mmu-mir-681 |
| mmu-mir-686 | mmu-mir-691 | mmu-mir-699 | mmu-mir-706 | mmu-mir-710 |
| mmu-mir-712 | mmu-mir-714 | mmu-mir-721 | mmu-mir-7a-1 | mmu-mir-7a-2 |
| mmu-mir-877 | mmu-mir-9-1 | mmu-mir-92a-1 | mmu-mir-93 | mmu-mir-99a |
| hsa-miR-424* | hsa-miR-605 | hsa-miR-34b | hsa-miR-663b | hsa-miR-202* |
| hsa-miR-663 | hsa-let-7b | hsa-mir-1-1 | hsa-mir-105-2 | hsa-mir-124-1 |
| hsa-mir-127 | hsa-mir-1307 | hsa-mir-135a-1 | hsa-miR-1305 | hsa-mir-135a-2 |
| hsa-miR-668 | hsa-mir-130a | hsa-mir-138-2 | hsa-mir-143 | hsa-mir-148a |
| hsa-mir-153-1 | hsa-mir-16-1 | hsa-mir-181b-2 | hsa-mir-185 | hsa-mir-190 |
| hsa-mir-194-1 | hsa-mir-197 | hsa-mir-19b-1 | hsa-mir-203 | hsa-mir-20b |
| hsa-mir-215 | hsa-mir-219-2 | hsa-mir-224 | hsa-miR-26a | hsa-mir-28 |
| hsa-mir-29c | hsa-mir-30a | hsa-mir-31 | hsa-mir-324 | hsa-mir-330 |
| hsa-mir-33a | hsa-mir-34a | hsa-mir-365-1 | hsa-mir-372 | hsa-mir-376b |
| hsa-mir-381 | hsa-mir-412 | hsa-mir-429 | hsa-mir-450a-1 | hsa-mir-483 |
| hsa-mir-488 | hsa-mir-494 | hsa-mir-500 | hsa-mir-509-1 | hsa-mir-512-2 |
| hsa-mir-516a-2 | hsa-mir-518a-1 | hsa-mir-518f | hsa-mir-519e | hsa-mir-520f |
| hsa-mir-523 | hsa-mir-527 | hsa-miR-550 | hsa-mir-571 | hsa-mir-584 |
| hsa-mir-604 | hsa-mir-625 | hsa-mir-637 | hsa-mir-649 | hsa-mir-660 |
| hsa-miR-7 | hsa-mir-766 | hsa-mir-891a | hsa-mir-92a-1 | hsa-mir-98 |
| mmu-mir-103-1 | mmu-mir-125a | mmu-mir-138-2 | mmu-mir-155 | mmu-mir-182 |
| mmu-mir-199b | mmu-mir-20b | mmu-mir-23b | mmu-mir-296 | mmu-mir-30e |
| mmu-mir-363 | mmu-mir-467a | mmu-mir-652 | mmu-mir-685 | mmu-mir-711 |
| mmu-mir-805 | | | | |

In some embodiments, the exosomes (natural or genetically engineered exosomes) comprise neuroblastoma-specific therapeutic micro RNAs including but not limited to micro RNA listed in Table 2.

TABLE 2
Exemplary neuroblastoma-specific micro RNAs (which refers to the-3p and the-5p mature
sequence of the indicated microRNA).

| | | | | |
|---|---|---|---|---|
| hsa-mir-34a | hsa-mir-181b-1 | hsa-mir-24-1 | hsa-mir-17 | hsa-mir-302a |
| hsa-mir-326 | hsa-mir-92a-1 | hsa-mir-335 | hsa-mir-141 | hsa-mir-24-1 |
| hsa-mir-30c-1 | hsa-let-7a-2 | hsa-mir-92a-1 | hsa-mir-129-2 | hsa-mir-99a |
| hsa-mir-149 | hsa-mir-200c | hsa-mir-199a-1 | hsa-mir-181b-2 | hsa-mir-323 |
| hsa-mir-93 | hsa-mir-190 | hsa-mir-106a | hsa-mir-330 | hsa-mir-184 |
| hsa-mir-27b | hsa-mir-30c-2 | hsa-let-7a-3 | hsa-mir-107 | hsa-mir-150 |
| hsa-mir-137 | hsa-mir-221 | hsa-mir-181a-2 | hsa-mir-216a | hsa-mir-331 |
| hsa-mir-181a-1 | hsa-mir-186 | hsa-mir-20a | hsa-mir-200a | hsa-mir-324 |
| hsa-mir-25 | hsa-mir-187 | hsa-mir-30b | hsa-let-7a-1 | hsa-mir-30e |
| hsa-let-7b | hsa-mir-129-1 | hsa-mir-181a-2 | hsa-mir-146a | hsa-mir-18a |
| hsa-mir-301a | hsa-mir-125a | hsa-mir-125b-1 | | |

In some embodiments, the exosomes (natural or genetically engineered exosomes) comprise leukemia-specific therapeutic micro RNAs including but not limited to the micro RNA listed in Table 3.

TABLE 3
Exemplary leukemia-specific micro RNAs (which refers to the-3p and the-5p mature
sequence of the indicated microRNA).

| | | | | |
|---|---|---|---|---|
| hsa-let-7a-1 | hsa-let-7a-3 | hsa-let-7b | hsa-let-7c | hsa-let-7d |
| hsa-let-7e | hsa-let-7f-1 | hsa-let-7f-2 | hsa-let-7g | hsa-let-7i |
| hsa-mir-101-1 | hsa-mir-103-1 | hsa-mir-106a | hsa-mir-106b | hsa-mir-107 |
| hsa-mir-1226 | hsa-mir-125b-1 | hsa-mir-126 | hsa-mir-128-1 | hsa-mir-1307 |
| hsa-mir-130a | hsa-mir-130b | hsa-mir-140 | hsa-mir-142 | mmu-mir-17 |
| mmu-mir-92a-1 | miR-15a | miR-16 | miR-155 | miR-21 |

In some embodiments, the exosomes (natural or genetically engineered exosomes) comprise miRNAs that regulated expression of key genes in neuroblastoma cells including but not limited to the miRNAs listed in Table 4.

TABLE 4
Exemplary miRNAs in aNK exosomes that may regulate expression of key genes in neuroblastoma cells.

| | | Target Gene | | | | | | | | |
|---|---|---|---|---|---|---|---|---|---|---|
| miRNA | Ct | MYCN | TGFBR1 | SMAD2 | SMAD3 | TGFB1 | TGFB2 | TGFB3 | ALK | AURKA |
| hsa-miR-17-002308 | 24.1 | X | X | X | X | | X | | | |
| hsa-miR-146a-000468 | 24.4 | | X | X | X | | | | | |
| hsa-miR-155-002623 | 24.6 | | X | X | X | | X | X | | |
| hsa-miR-222-002276 | 24.8 | | X | X | | | X | | | |
| hsa-miR-16-000391 | 25.4 | | X | X | X | | X | | | |
| hsa-miR-19b-000396 | 25.5 | X | X | X | | | X | | | |
| hsa-miR-484-001821 | 25.7 | X | X | X | X | | | | | X |
| hsa-miR-146b-001097 | 25.9 | | X | X | X | | X | X | | X |
| hsa-miR-24-000402 | 26.0 | | X | X | | | X | | X | X |
| hsa-miR-150-000473 | 26.5 | X | X | | X | | X | | | |
| hsa-miR-342-3p-002260 | 26.8 | | | X | X | | | X | | |
| hsa-miR-186-002285 | 27.2 | X | X | X | X | | X | X | X | X |
| hsa-miR-29a-002112 | 27.3 | X | | X | X | | X | X | | |
| hsa-miR-20a-000580 | 27.4 | X | X | X | X | | X | | | |
| hsa-miR-618-001593 | 27.7 | | X | X | X | | X | | | |
| hsa-miR-411-001610 | 27.8 | | | X | X | | X | | | |
| hsa-miR-142-3p-000464 | 28.0 | | X | X | | X | X | X | | |
| hsa-miR-320-002277 | 28.1 | | X | X | X | | X | | | |
| hsa-miR-518b-001156 | 28.4 | | X | | | | | | | |
| hsa-miR-19a-000395 | 28.5 | X | X | | | | X | | | |
| hsa-miR-454-002323 | 28.5 | | | X | | X | X | | | |
| hsa-miR-223-002295 | 28.7 | | | X | X | | | | | |

In some exemplary embodiments, the exosomes derived from aNK cells comprise mRNAs as shown in Table 5.

TABLE 5
Exemplary NK associated mRNAs in aNK
associated exosomes as determined by
TLDA assay. Ct = cycle threshold
(lower value is larger amountof mRNA).

| mRNA | Ct |
|---|---|
| GNLY-Hs00246266_m1 | 22.0 |
| B2M-Hs99999907_m1 | 23.0 |
| GAPDH-Hs99999905_m1 | 24.9 |
| CD14-Hs02621496_s1 | 27.2 |
| CXCR4-Hs00607978_s1 | 27.9 |
| IL8-Hs00174103_m1 | 28.7 |
| FCGR3B; FCGR3A-Hs00275547_m1 | 28.7 |
| KLRK1-Hs0018368_m1 (NKG2D) | 28.8 |
| TGFB1-Hs00171257_m1 | 28.8 |
| HPRT1-Hs99999909_m1 | 29.6 |
| IL2RA-Hs00166229_m1 | 30.1 |
| IFNG-Hs00174143_m1 | 31.6 |
| CD19-Hs00174333_m1 | 31.6 |
| CD8A-Hs00233520_m1 | 31.9 |
| CD86-Hs00199349_m1 | 32.6 |
| GZMB-Hs00188051_m1 | 33.0 |
| IL13-Hs00174379_m1 | 33.0 |
| CD40LG-Hs00163934_m1 | 33.1 |
| TBX21-Hs00203436_m1 | 33.2 |
| SDHA-Hs00417200_m1 | 33.2 |
| HMOX1-Hs00157965_m1 | 33.5 |
| CTLA4-Hs00175480_m1 | 33.9 |
| CSF1-Hs00174164_m1 | 34.3 |
| NCAM1-Hs00169851_m1 | 35.4 |
| CXCR3-Hs00171041_m1 | 35.6 |
| CX3CR1-Hs00365842_m1 | 35.6 |

In various embodiments, additional therapies may be prescribed with the compositions described herein comprising any one or more of (i) ex vivo expanded cells that naturally produce Fc receptors, (ii) ex vivo expanded cells that are engineered to produce Fc receptors (iii) ex vivo expanded aNK cells, (iv) natural exosomes obtained from ex vivo expanded cells that naturally produce Fc receptors, (v) engineered exosomes obtained from ex vivo expanded cells that are engineered to produce Fc receptors, (vi) exosomes obtained from ex vivo expanded aNK cells, (vii) genetically modified exosomes (either obtained from ex vivo expanded autologous genetically modified cells or exosomes that are modified after obtaining exosomes from ex vivo expanded genetically modified cells), or combinations thereof. In various embodiments, the therapy may be any one or more of surgery, radiation, chemotherapy, immunotherapy, vaccine or combinations thereof.

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, therapies include, for example, radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, therapies include, for example, immunotherapy. Immunotherapy may comprise, for example, use of cancer vaccines and/or sensitized antigen presenting cells. In some embodiments, therapies include targeting cells in the tumor microenvironment or targeting immune cells. For example, T-cells may be activated by administering a composition comprising the exosomes described herein that include a PD-1 (programmed death 1) CAR or macrophages may be downregulated by administering a compositions comprising exosomes described herein that include, for example, micro RNAs that target regulatory genes in the macrophages. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, therapies include, for example, hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

The duration and/or dose of treatment with anti-cancer therapies may vary according to the particular anti-cancer agent or combination thereof. An appropriate treatment time for a particular cancer therapeutic agent will be appreciated by the skilled artisan. The invention contemplates the continued assessment of optimal treatment schedules for each cancer therapeutic agent, where the genetic signature of the cancer of the subject as determined by the methods of the invention is a factor in determining optimal treatment doses and schedules.

In various embodiments, the subject for whom predicted efficacy of an anti-cancer therapy is determined, is a mammal (e.g., mouse, rat, primate, non-human mammal, domestic animal such as dog, cat, cow, horse), and is preferably a human. In another embodiment of the methods of the invention, the subject has not undergone chemotherapy or radiation therapy. In alternative embodiments, the subject has undergone chemotherapy or radiation therapy. In related embodiments, the subject has not been exposed to levels of radiation or chemotoxic agents above those encountered generally or on average by the subjects of a species. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient, or e.g., the subject is given the anti-cancer therapy prior to removal of the cancerous tissue.

In various embodiments, an effective amount of the one or more compositions is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages of an effective amount of the one or more composition can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, such as biopsied malignant tumors, or the responses observed in the appropriate animal models. In various embodiments, the compositions of the invention comprising the retinoid agonist may be administered once a day (SID/QD), twice a day (BID), three times a day (TID), four times a day (QID), or more, so as to administer an effective amount of the retinoid agonist to the subject, where the effective amount is any one or more of the doses described herein.

In some embodiments, the exosomes described herein may be used to deliver imaging agents to a subject in need thereof by administering to the subject a composition comprising the exosomes and one or more imaging agents including but not limited to F-18, F-19, Tc-99m or 1-123. In some embodiments, for pre-clinical imaging, near infrared fluorescent dyes (for example, Xenolight DiR) may be added to NK cells or to the exosomes.

### Kits of the Invention

The invention also provides a kit to treat, cancer, prevent metastasis of cancer, reduce the severity of cancer, promote remission of cancer and/or prevent or reduce the likelihood of cancer relapse in a subject in need thereof. The kit comprises a pharmaceutical composition described herein and instructions for use of the composition for treating cancer, preventing cancer, reducing the severity of cancer, promoting remission of cancer and/or prevent or reduce the likelihood of cancer relapse in subjects in need thereof. In some embodiments, the cancer is neuroblastoma. In some embodiments, the cancer is ALL or other leukemia subtypes. The kit is an assemblage of materials or components, including at least one of the compositions described herein.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome, such as so treat, inhibit, reduce the symptoms of and/or promote prophylaxis of autoimmune diseases and/or cancer in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of a composition described herein. The packaging material generally has an external label which indicates the contents and/or purpose of the kit and/or its components.

### Therapeutic Efficacy

Also provided herein are methods determining the efficacy of antibody dependent exosome therapy comprising determining the targeting efficacy of the exosomes to the target cells and/or tissues. In some embodiments, determining the targeting efficacy of the exosomes to the target cells and/or tissues includes detecting in the target cells and/or tissues the nucleic acids and/or proteins that are specific to the exosomes. The presence of exosome-specific nucleic acids and/or proteins in the target cells and/or tissues is indicative of effective antibody dependent exosome therapy. In some embodiments, the exosomes are derived from activated natural killer cells. In some embodiments, the exosomes are engineered to include specific proteins and/or nucleic acids that are unique to the exosomes. In some embodiments, nucleic acids include miRNAs, mRNAs or a combination thereof. In exemplary embodiments, the miRNAs may be any one or more of the miRNAs set forth in Table 4. In further exemplary embodiments, the mRNAs may be any one or more of the mRNAs set forth in Table 5.

Techniques that may be used to assess the amount of exosome-specific nucleic acid in the target cells and/or tissues, include but are not limited to in situ hybridization (e.g., Angerer (1987) Meth. Enzymol 152: 649). Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches. Probes that may be used for nucleic acid analysis are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel, et al. (1998) Nature Genetics 20: 207-211, and/or Kallioniemi (1992) Proc. Natl Acad Sci USA 89:5321-5325 (1992).

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Additional methods for determining the presence of exosome-specific proteins and nucleic acids in target cells and/or tissues include but are not limited to TaqMan PCR for nucleic acids, immunofluorescence or flow cytometry for proteins.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

We have a method to produce abundant quantities of NK exosomes in vitro that can be specifically directed against a neuroblastoma-specific cell surface marker called GD2. This could be achieved by transfecting NK cells with an anti-GD2 CAR. Alternatively, the NK-derived exosomes could be coated with an anti-GD2 antibody that therefore delivers the exosomes mainly (if not exclusively) to neuroblastoma cells expressing GD2 on their surface. Therapeutic agents that could increase killing of cancer cells by the exosomes include more perforin or granzyme and microRNAs (miRNAs) that regulate gene expression in the cancer cell. It is possible to overexpress a specific miRNA of interest in NK cells and hence in their exosomes so that they kill the cancer cells or make them more susceptible to other treatments (e.g., “miRNA-enriched” anti-neuroblastoma exosomes).

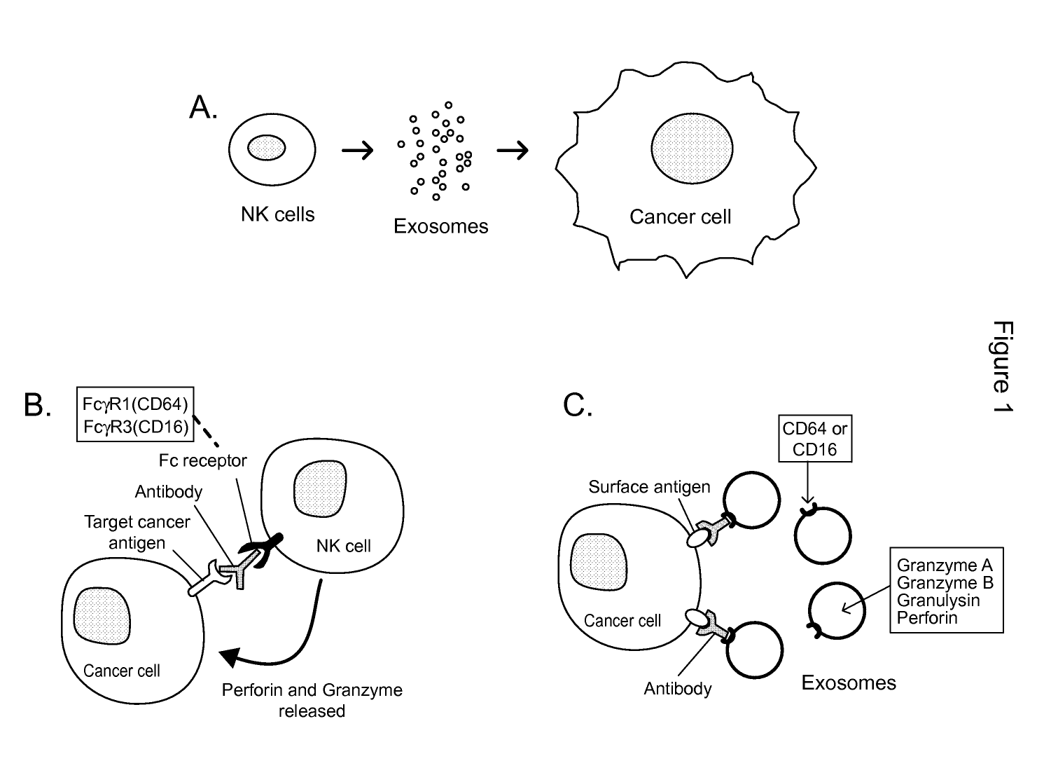

FIG. 1 is a schematic depiction of the release of exosomes by NK cells, antibody dependent cellular cytotoxicity (ADCC), and antibody dependent exosome cytotoxicity (ADEC) mediated by exosomes released from NK cells.

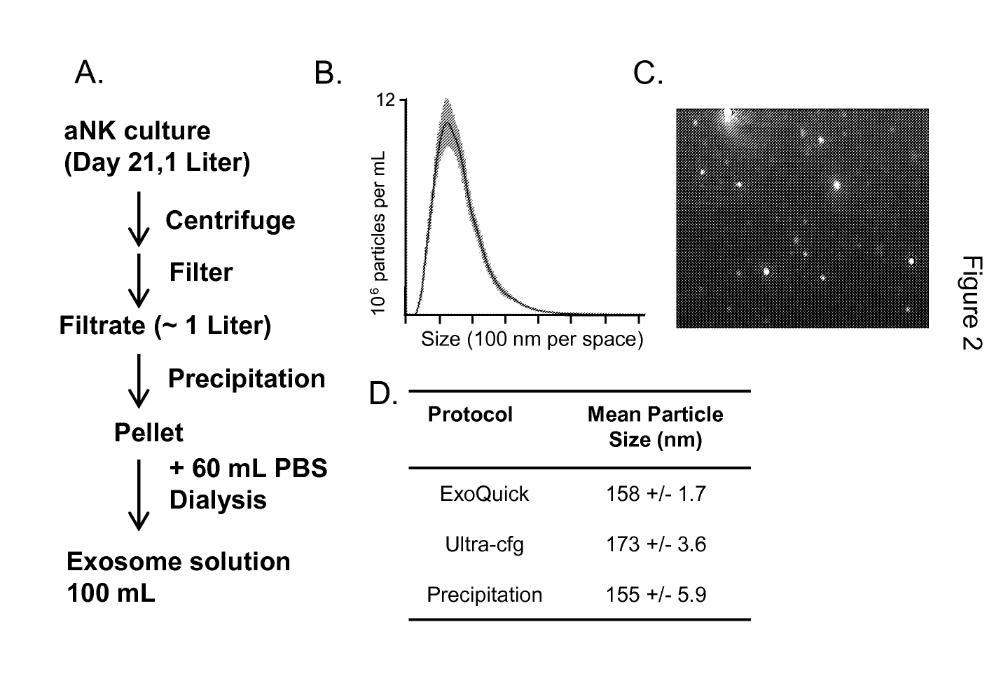

FIG. 2 describes, in an exemplary embodiment, ex vivo expansion of activated Natural Killer cells and large scale aNK exosome isolation.

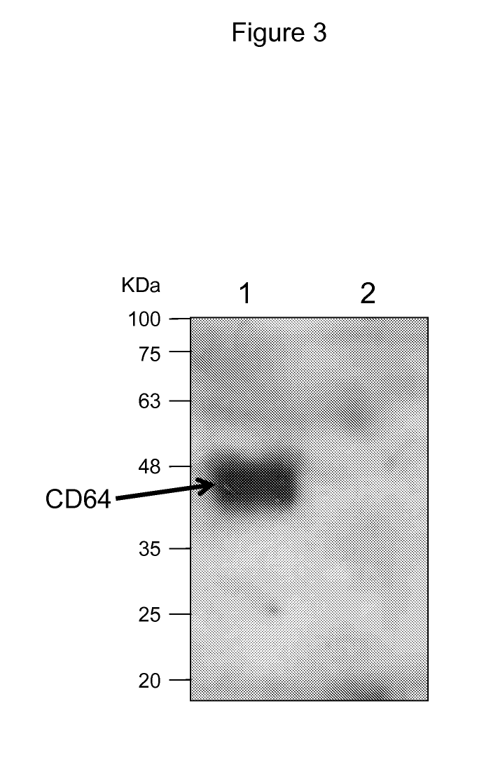

FIG. 3 is a Western blot showing that activated NK cells derived exosomes express CD64. Mass spectrometry data also showed that activated NK cells derived exosomes express CD64.

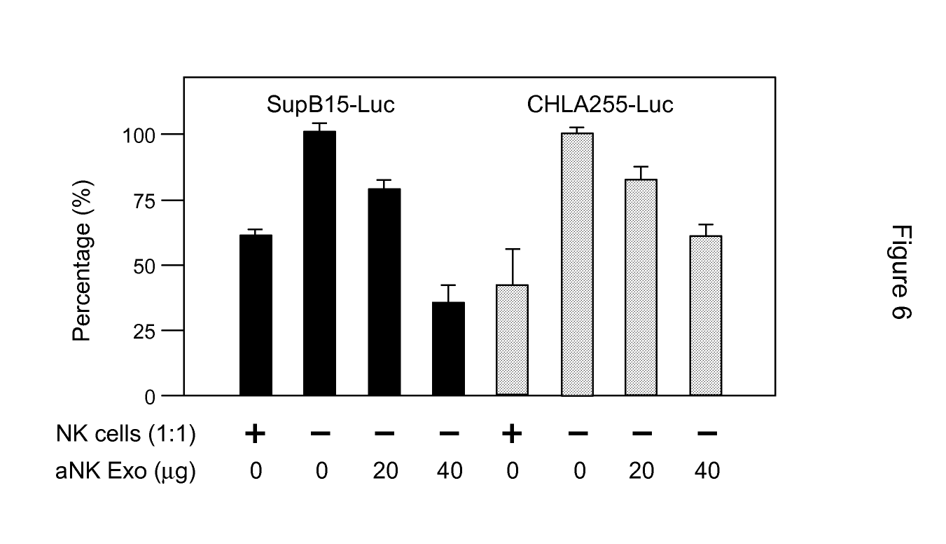

Infusions of natural killer (NK) cells are an emerging tool for cancer immunotherapy. The method to produce large numbers of fully functional aNK cells is a critical step to this approach (Liu Y, et al., Growth and activation of natural killer cells ex vivo from children with neuroblastoma for adoptive cell therapy. Clin Cancer Res. 2013; 19(8):2132-43; Seeger R C. Immunology and immunotherapy of neuroblastoma. Seminars in cancer biology. 2011; 21(4):229-37; Denman C J, et al., Membrane-Bound IL-21 Promotes Sustained Ex Vivo Proliferation of Human Natural Killer Cells. PloS one. 2012; 7(1):e30264. PMCID: 3261192), and our recent study has shown that co-culturing patient PBMC with K562mbIL-21 aAPC can induce major expansion (>2,000 fold) of aNK cells, which provides sufficient aNK cells for autologous clinical treatments. We extend this approach to produce exosomes from aNK cells. We also have the necessary in vitro and in vivo pre-clinical neuroblastoma models for the proposed research. Our experiments demonstrate 1) more than 10 mg quantities of aNK exosomes can be produced and purified; and 2) purified aNK exosomes from two different donors are cytotoxic for neuroblastoma and ALL cell lines (FIG. 6).

Exosomes were isolated from 48-72 hour conditioned medium of aNK cells by ultra-centrifugation and proprietary polymer precipitation methodologies. Neuroblastoma CHLA255-Luc cells or SupB15-Luc ALL cells (10⁴ cells) were incubated in 96 well plates with aNK cells (10⁴ cells; effector to target cell ratio 1:1) or different amounts of purified exosomes as indicated for 6 hours. Luciferase substrate Beetle Luciferin (Promega, E1605) was added, and then bioluminescence was quantified. Untreated samples (FIG. 6 lanes 2 or 6) were designated as 100% for CHLA255-Luc or SupB15-Luc, respectively. Luc, firefly luciferase labeled cells.

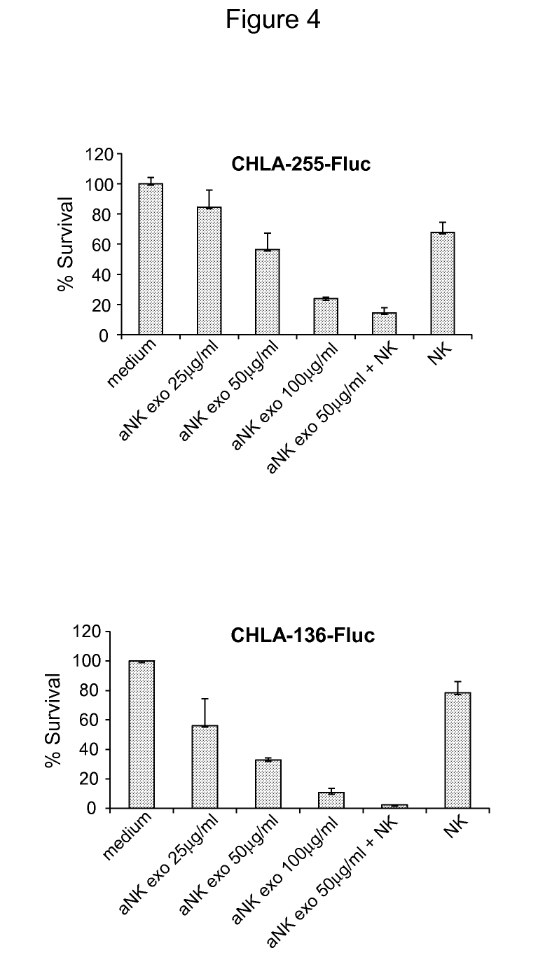

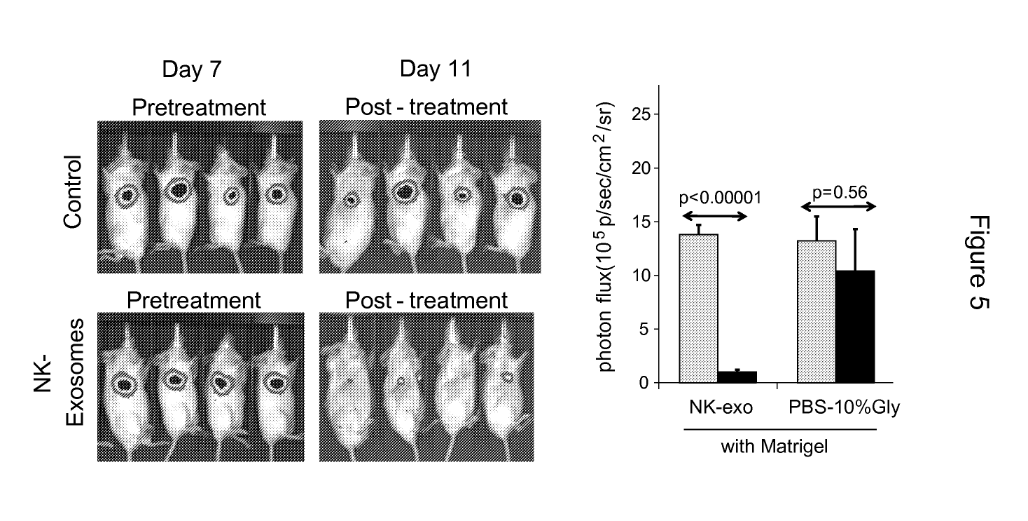

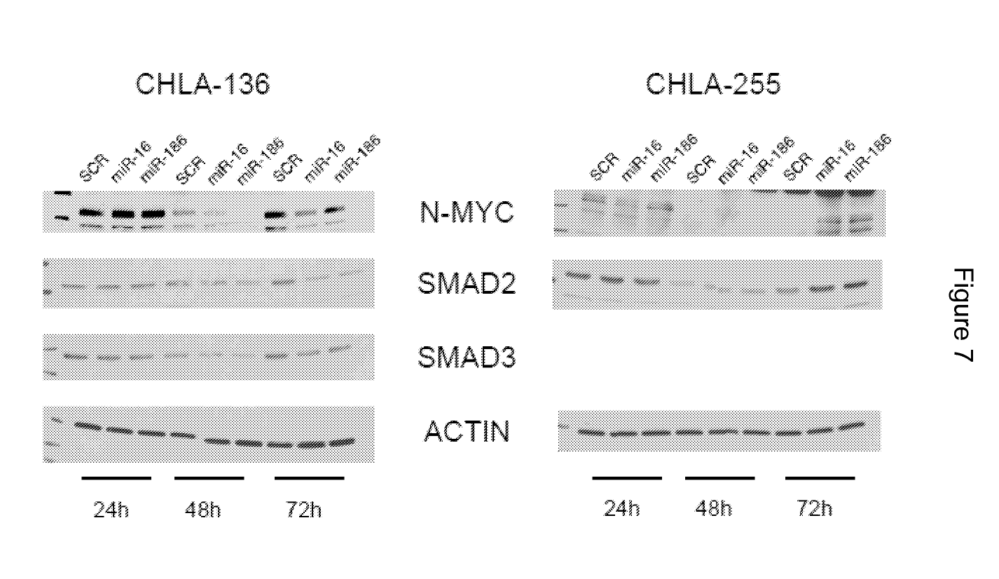

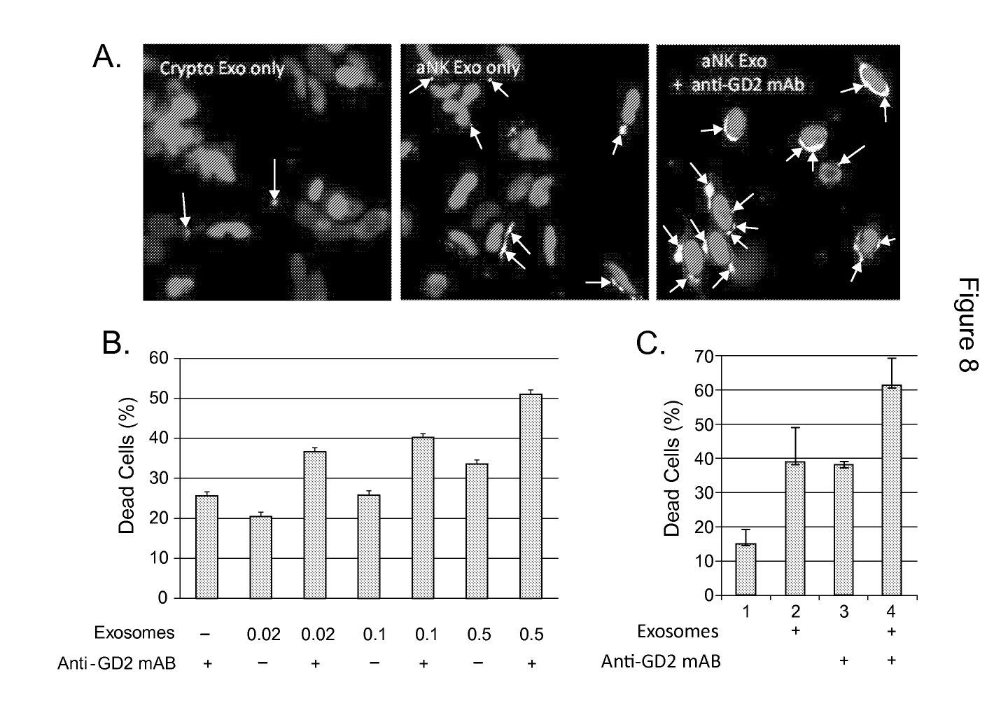

As shown in FIG. 4, aNK exosomes increase cytotoxicity of aNK cells. Further aNK-derived exosomes are cytotoxic for NB cells in NOD/SCID mice (FIG. 5). As described herein, the exosomes described herein may be engineered to include therapeutic miRNAs and/or antibodies. Protein expression of MYCN and SMAD2/3 is suppressed by miR-16 and miR-186 in human neuroblastoma cell lines (FIG. 7). Anti-GD2 monoclonal antibodies increase interaction between aNK exosomes (white arrows) and neuroblastoma cells (grey) (FIG. 8).

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A pharmaceutical composition comprising exosomes comprising an Fc receptor and a pharmaceutically acceptable carrier.

2. The pharmaceutical composition of claim 1, wherein the Fc receptor is any one or more of FcγR1(CD64), FcγR2(CD32), FcγR3(CD16) or combinations thereof.

3. The pharmaceutical composition of claim 1, wherein the exosomes are natural exosomes.

4. The pharmaceutical composition of claim 3, wherein the exosomes are derived from activated natural killer cells.

5. The pharmaceutical composition of claim 1, wherein the exosomes are engineered exosomes.

6. The pharmaceutical composition of claim 5, wherein the engineered exosomes comprise FcγR1(CD64), FcγR2(CD32), FcγR3(CD16) or combinations thereof.

7. The pharmaceutical composition of claim 1, further comprising one or more antibodies, wherein the antibodies target the exosomes to the cancer cells.

8. The pharmaceutical composition of claim 15, further comprising one or more therapeutic agents, wherein the therapeutic agent is any one or more of lipids, peptides, proteins, antibodies, small molecules, oligonucleotides, nucleic acids or a combination thereof.

9. (canceled)

10. A method for treating cancer in a subject in need thereof comprising:
providing a composition comprising exosomes derived from cells, wherein the exosomes comprise Fc receptors; and
administering an effective amount of the composition to the subject so as to treat cancer in the subject.

11. The method of claim 10, wherein the cancer is any one or more of lymphomas, sarcomas, brain cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer.

12. The method of claim 10, wherein the cancer is neuroblastoma or leukemia.

13. The method of claim 12, wherein the leukemia is ALL.

14. The method of claim 10, wherein the composition comprises natural exosomes obtained from cells that produce Fc receptors or engineered exosomes obtained from cells that are engineered to produce Fc receptors.

15. (canceled)

16. The method of claim 14, wherein the Fc receptor is any one or more of FcγR1(CD64), FcγR2(CD32), FcγR3(CD16) or combinations thereof.

17. The method of claim 14, wherein the cells are anyone or more of activated natural killer (aNK) cells, T cells, macrophages, stem cells, mesenchymal stromal cells or combinations thereof.

18. The method of claim 10, wherein the composition further comprise one or more antibodies, wherein the antibodies target the exosomes to the cancer cell.

19. The method of claim 18, wherein the antibodies target any one or more of GD2, CD24, CD25 or a combination thereof on the surface of neuroblastoma cells.

20. The method of claim 18, wherein the antibodies target any one or more of CD2, CD10, CD15, CD19, CD21, CD22 or a combination thereof.

21. The method of claim 10, wherein the composition further comprise one or more therapeutic agents, wherein the therapeutic agent is any one or more of lipids, peptides, proteins, antibodies, small molecules, oligonucleotides, nucleic acids or a combination thereof.

22. (canceled)

23. The method of claim 22, wherein the therapeutic agents are any one or more of anti-GD2 antibodies, 3F8 antibody, recombinant GM-CSF or a variant thereof, irinotecan/SN38, etoposide, doxorubicin, fenretinide, PI-103, cyclophosamide or a combination thereof.

24-27. (canceled)
